(19) United States
(12) Patent Application Publication
(10) Pub. No.: US 2020/0146532 A1
(43) Pub. Date: May 14, 2020

(54) IMAGING UNIT FOR A SURGICAL INSTRUMENT, AND METHOD FOR MANUFACTURING AN IMAGING UNIT

(71) Applicant: OLYMPUS WINTER & IBE GMBH, Hamburg (DE)

(72) Inventor: Uwe SCHOELER, Hoisdorf (DE)

(73) Assignee: OLYMPUS WINTER & IBE GMBH, Hamburg (DE)

(21) Appl. No.: 16/733,827

(22) Filed: Jan. 3, 2020

Related U.S. Application Data
(63) Continuation of application No. PCT/EP2018/068614, filed on Jul. 10, 2018.

(30) Foreign Application Priority Data
Jul. 24, 2017 (DE) 10 2017 116 652.1

(51) Int. Cl. A61B 1/00

(52) U.S. Cl. CPC A61B 1/00096; A61B 1/00128; A61B 1/0011

(57) ABSTRACT

An imaging unit for a surgical instrument. The imaging unit including: an objective tube; and an optical element accommodated in the objective tube, wherein one or more of: the optical element is clamped gap-free in the objective tube; the optical element is accommodated in a mount in the optical tube and the mount is clamped gap-free in the objective tube; and the optical element is accommodated in a centered mount.

### CROSS-REFERENCE TO RELATED APPLICATION

The present application is a continuation of PCT/EP2018/068614 filed on Jul. 10, 2018, which is based upon and claims the benefit to DE 10 2017 116 652.1 filed on Jul. 24, 2017, the entire contents of each of which are incorporated herein by reference.

### BACKGROUND

### Field

The present disclosure relates to an imaging unit for a surgical instrument, such as an endoscope. Moreover, the present disclosure relates to a surgical instrument, such as an endoscope. Furthermore, the present disclosure also relates to a method for manufacturing an imaging unit for a surgical instrument, such as an endoscope.

### Prior Art

Continuously rising requirements are being placed on the image quality of optical systems like those used in surgical instruments, such as in endoscopes. It was previously sufficient to polish the optical components and adjust their outer diameter with a relatively large tolerance. To install the optical system, the optical components are arranged in a system tube such as in an objective tube. There is a clearance fit in this case between the optical elements and the system tube. In the direction of an optical axis of the system, so-called aperture tubes that fix the optical components at a distance from each other in the system tube are located between the optical elements.

The provided clearance fit provides a certain amount of play, however, for the individual optical components in a radial direction of the system tube. Furthermore, the optical components can shift axially slightly or tilt relative to the optical axis of the system. Such deviations from the ideal adjustment can negatively affect the image quality of the optical system.

In optical imaging systems, such as video endoscopes, the lens is focused by transverse displacement, i.e., by a displacement along its optical axis, relative to a plane in which a sharp image is desired and in which e.g. an image sensor is located. Then the lens, such as the endoscope lens, is permanently fixed by e.g. being glued. In practice, primarily rotationally symmetrical cylindrical fits are used to align the optical components. Despite minimal tolerances in the fit, it is possible for the optical element or the optical unit to tilt slightly. Continuously rising demands are being placed on the coaxiality between a normal of the image sensor and the optical axis of the image-generating optical element which, for example, is part of an endoscope lens, especially in high-resolution optical units, so that the achievable image quality can be fully exploited.

To satisfy these high demands, it would be possible to increase a guide length in the fits. However, this simultaneously leads to a loss of light and possibly greater dispersion. Further reducing the fit tolerances only theoretically allows a potential tilt to be reduced since there must always be a minimum play in the fit to install the optical element.

For example, an imaging unit for an endoscope is known from DE 10 2015 205 457 A1.

### SUMMARY

An object is to present an imaging unit, an endoscope, and a method for manufacturing an imaging unit, wherein the imaging unit enables a precise alignment of the optical element.

Such object can be achieved by an imaging unit for a surgical instrument, such as an endoscope, having an optical element, such as an optical lens, wherein the optical element is accommodated in an objective tube, wherein a.) the optical element is clamped gap-free in the objective tube, or b.) the optical element is accommodated in a mount, and the mount of the optical element is clamped gap-free in the objective tube, and/or the optical element is accommodated in a centered mount.

After introducing the mount with the optical element, such as the optical lens, the mount is held and aligned in the objective tube by a clamping between the mount and the objective tube, wherein the mount is arranged gap-free in the objective tube due to the clamping of the mount in the objective tube, whereby the optical lens is aligned as an optical element in the objective tube without tilting relative to the optical axis. In this case, the optical element is accommodated in a centered mount so that the mount for the lens as an optical element has a precise outer diameter, and the optical axis of the lens is aligned collinearly, i.e., overlaps, with the mechanical axis of the mount.

In order to provide a centered mount with a lens for an objective tube, the mount is clamped with an e.g. glued lens in an adjusting chuck so that afterwards, the position of the optical axis of the lens relative to the spindle axis is detected and measured. By means of the adjusting chuck, the lens with the mount is subsequently aligned so that its two curvature midpoints lie as precisely as possible on the rotational axis of the spindle. Then the adjusting spindle is rotated, and the outer surface of the mount, for example made of stainless steel, is for example processed with a turning tool. This gives rise to a precisely machined surface of the mount that is aligned parallel to the spindle axis. A centered mount with a lens is thereby formed, or respectively provided.

Before machining, the lens is fixed in the mount, for example by means of low stress adhesives. Furthermore, the lenses can be arranged in the mount to be flanged or held by threaded rings.

For the gap-free arrangement of the mount with the optical element in the objective tube of the imaging unit, the centered mount can have a precise diameter that is smaller than the diameter of the objective tube while being inserted or introduced into the mount. After arranging the mount in the objective tube, the inner diameter of the objective tube is constricted so that the mount is clamped gap-free as a result of the constriction of the (inner) diameter of the objective tube.

For example, the objective tube can be produced from stainless steel and, before the centered mount is introduced, formed with a gap running in the longitudinal direction of the objective tube, wherein the inner diameter of the objective tube is greater than the outer diameter of the mount. After arranging the mount formed with the lens accommodated in the interior in the objective tube, the gap is at least partially closed, for example by welding, or respectively a weld seam, whereby the inner diameter of the objective tube is also reduced, and thereby the mount in the objective tube is accordingly clamped.

Alternatively, the optical element can be held and aligned without a mount after introducing the optical element, such as the optical lens, by a direct clamping between the optical element and the objective tube, wherein, due to the clamping of the optical element in the objective tube without an intermediate mount for the optical element, the optical element, such as the lens, is arranged gap-free in the objective tube, whereby the optical lens is aligned in the objective tube as an optical element without tilting relative to the optical axis.

The objective tube can have a through-hole in the region of the optical element, wherein when the optical element is arranged in the objective tube in the region of the through-hole, the optical element is not brought into contact with an inner lateral surface of the objective tube that faces the optical element, or the objective tube has a through-hole in the region of the optical element and/or in the region of the mount, wherein when the mount with the optical element is arranged in the objective tube in the region of the through-hole, the mount of the optical element is not brought into contact with an inner lateral surface of the objective tube that faces the mount with the optical element. Outside of the through-hole in the objective tube and outside of the region of the mount, i.e., in the region adjacent to the mount in the objective tube and/or in the region adjacent to the through-hole, the inner diameter of the objective tube can have at least the same as or less than the outer diameter of the centered mount so that the mount is clamped gap-free.

The through-hole in the objective tube can be formed as a gap or slot, wherein the gap or the slot extends in the longitudinal direction of the objective tube relative to the longitudinal extension of the objective tube. In one embodiment, the gap or the slot is a cut in the objective tube of stainless steel using a laser such as an Nd-YAG laser. In one alternative, the gap or slot can be formed by an erosion method or the like in the objective tube. Typically, the width of the gap or the slot is between 30 to 100 μm.

Moreover, the through-hole in the objective tube for the optical element can be formed between two slot connecting regions of the objective tube relative to the longitudinal extension of the objective tube.

In this regard, one embodiment provides that at least one of the two slot connecting regions is formed as a weld seam. For example, the gap in the objective tube is welded, and thereby partially closed, such as by a pulsed laser, such as an Nd-YAG laser, up to the through-hole after introducing the centered mount into the objective tube, wherein the inner diameter of the objective tube is restricted, or respectively decreased upon welding the gap.

Since the weld seam is interrupted by the through-hole in the region of the mount, it is possible to introduce or feed adhesives into the through-hole in order to thereby glue the objective tube arranged in the sleeve tube to the sleeve tube upon arranging the objective tube in a sleeve tube for the objective tube. For example, the sleeve tube can be formed with an image sensor such as a CMOS chip on a head side, as well as a guide tube for the objective tube. The sleeve tube is arranged in an endoscope shaft of an endoscope so that images are detected by means of the image sensor and shown on a monitor or the like.

The weld seam or the weld seams can be formed by laser welding.

Such object can be furthermore achieved by an imaging unit for a surgical instrument, such as an endoscope, with a sleeve tube having two head ends to accommodate an objective tube accommodating at least one optical element, such as an optical lens, wherein the head ends of the sleeve tube are formed tubular, and the two head ends of the sleeve tube are connected to each other by means of at least one, connecting bar, which can be flexible.

The sleeve tube can be provided at one head end or in the region thereof with an image sensor such as a CMOS chip, CCD chip or the like in order to detect the images transmitted by a lens system. Since the sleeve tube is formed with one or more connecting bars between the two tubular head ends, it is possible, when arranging an objective tube that can be formed as described above, to adjust and/or fix the objective tube in the sleeve tube by bending the connecting bar or connecting bars. In so doing, the connecting bar or the connecting bars are brought into contact with the peripheral surface of the objective tube. By adjusting the objective tube in the sleeve tube, a tilt of the optical axis relative to the optical axis of an image sensor is eliminated or nearly overcome. The sleeve tube can be formed as a guide tube for the objective tube.

In this regard, one embodiment of the imaging unit provides that the head ends of the sleeve tube are connected to each other by means of a plurality of connecting bars, which can be flexible, wherein the connecting bars can be arranged evenly and/or symmetrically in the peripheral direction of the sleeve tube. Accordingly, the connecting bars surround the objective tube inserted in the sleeve tube.

A gap or a recess can be formed in the peripheral direction of the sleeve tube between two adjacent connecting bars. In this case it is possible for the connecting bars to be formed or arranged between the two end-side tube sections, or respectively the tubular head ends of the sleeve tube, and to connect the end-side tube sections, or respectively the tubular head ends with each other. An image sensor can be arranged on or in a head end of the sleeve tube, whereas the other tubular head end is open so that the objective tube can be introduced into the sleeve tube through this second head end.

Moreover, the imaging unit is distinguished in that the width of one of the connecting bar or of the connecting bars can be greater in the peripheral direction of the sleeve tube than the width of the space or the width of the recess between two adjacent connecting bars.

In addition, the connecting bar or the connecting bars can be flexible or curved in a radial direction relative to the longitudinal axis of the sleeve tube, such as to the longitudinal axis.

Moreover, one embodiment of the imaging unit provides that an objective tube with an optical element, such as an optical lens, can be accommodated in the sleeve tube, wherein the optical element can be arranged in a centered mount, and the mount can be clamped gap-free in the objective tube. Such an objective tube is described above, wherein reference is expressly made to the above statements.

Moreover, such object can be achieved with a surgical instrument, such as an endoscope, that is configured with the above-described imaging unit. In this case, the imaging unit can be provided with an objective tube having a mount accommodated therein for an optical element, such as an optical lens, and/or having a sleeve tube for an objective tube arranged or to be arranged therein. To avoid repetition, explicit reference is made to the statements above.

Such object can be furthermore achieved by a method for manufacturing an imaging unit for a surgical instrument, such as an endoscope, wherein the imaging unit has a sleeve tube and an objective tube arranged in the sleeve tube, wherein an optical element, in particular an optical lens, is accommodated in the objective tube, the method comprising:

a.) the optical element is inserted into the objective tube provided with a longitudinal slot, or the optical element is accommodated in a mount and is inserted with the mount into the objective tube provided with a longitudinal slot,

b.) after inserting the optical element, or after inserting the optical element accommodated in the mount, into the objective tube, the diameter, such as the inner diameter, of the objective tube is reduced, and/or the optical element or the mount of the optical element is clamped, such as being clamped gap-free in the objective tube, and

c.) then the objective tube is introduced, such as being shoved into the sleeve tube.

The produced imaging unit can be provided with the above-described objective tube and can also be provided with the sleeve tube that are described above in detail. After the production of the imaging unit, it can be arranged in an endoscope shaft of an endoscope. By means of an optical measuring device, the optical axis of the optical element is detected so that, by bending the connecting bar or the connecting bars, the tilt of the optical axis of the optical element is eliminated, depending on the measuring results, relative to the optical axis of the image sensor, or respectively with regard to the longitudinal axis of the sleeve tube.

In one embodiment of the method, after the optical element is inserted into the objective tube, the longitudinal slot of the objective tube can be closed partially by welding in the longitudinal direction, such as welded, so that the diameter, such as the inner diameter, of the objective tube is reduced, and/or the optical element or the mount of the optical element is clamped.

Moreover, the longitudinal slot of the objective tube is not welded in the region of the optical element, and is welded outside of the region of the optical element, or the longitudinal slot of the objective tube is welded in the region of the mount of the optical element, and is welded outside of the region of the mount.

The objective tube can be introduced into the sleeve tube, wherein the sleeve tube has two tubular head ends for the objective tube, and the two head ends of the sleeve tube are connected to each other by at least one connecting bar, which can be flexible, or by means of a plurality of connecting bars, which can be flexible.

The imaging unit can be formed with an objective tube and sleeve tube described above. In this regard, reference is additionally made to the above statements.

Further features will become apparent from the description of the embodiments together with the claims and the attached drawings. Embodiments can fulfill individual features or a combination of several features.

### BRIEF DESCRIPTION OF THE DRAWINGS

The embodiments are described below, without restricting the general idea of the invention, using exemplary embodiments with reference to the drawings, express reference being made to the drawings with regard to all details that are not explained in greater detail in the text. In the following:

FIG. 1 illustrates an endoscope in a schematically simplified side view;

FIG. 2a schematically illustrates a simplified longitudinal section of an objective tube for an imaging unit of an endoscope;

FIG. 2b schematically illustrates a longitudinal section of an imaging unit of an endoscope having the objective tube illustrated in FIG. 2a;

FIG. 2c schematically illustrates a simplified longitudinal section of an objective tube for an imaging unit of an endoscope according to another embodiment;

FIG. 3a schematically illustrates a simplified longitudinal section of a guide tube of an imaging unit of an endoscope for an objective tube, and

FIG. 3b schematically illustrates a simplified cross-sectional view along intersection line B-B in FIG. 3a.

In the drawings, the same or similar elements and/or parts are provided with the same reference numbers in order to prevent the item from needing to be reintroduced.

### DETAILED DESCRIPTION

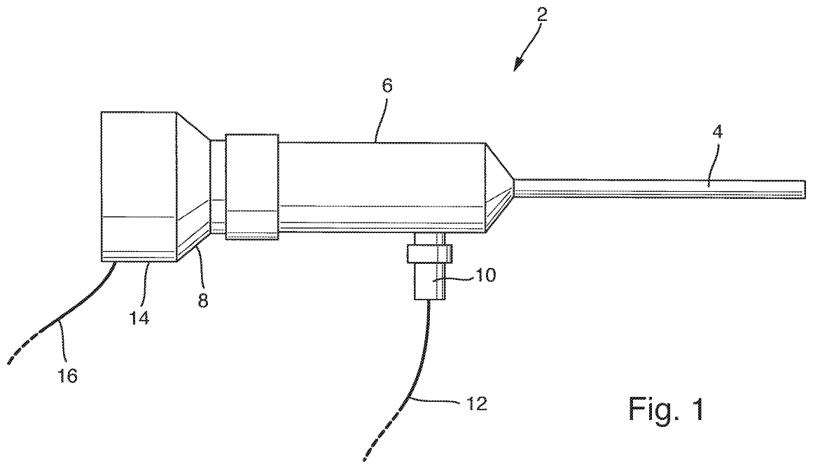

FIG. 1 shows a schematic and simplified side view of a preferably rigid endoscope 2 such as a video endoscope. On its distal end, the endoscope 2 comprises a tubular shaft 4 in which an optical element such as an endoscope lens is arranged. With the assistance of the endoscope lens, a surgical and investigative region is observed, or respectively depicted, which lies distally in front of a free end of the shaft 4. Starting from the endoscope lens, the image is passed on optically or electronically through the shaft 4 which terminates in a housing 6.

At the proximal end of the endoscope 2 is a housing 6 with an eyepiece 8. The housing 6 serves for handling the endoscope 2. On this side of the housing 6 is a light source 10, such as an LED light source. This is connected by a connecting cable 12 to a suitable power supply. In the configuration of a video endoscope, a handle as well as a video unit can be provided.

A schematically portrayed camera head 14 with an ocular adapter (not shown) is arranged on the eyepiece 8. The camera head 14 detects the light exiting the endoscope 2 with an image sensor. The camera head 14 is supplied with power by means of a connection 16. Furthermore, it is possible to send image signals by the connection 16 from the surface sensor of the camera head 14 to an external evaluation unit and transmit control signals to the camera head 14. Whereas a CCD sensor is not provided in the design of a surgical instrument with a relay lens system, a video endoscope has corresponding lenses and for example a CCD sensor as an image sensor.

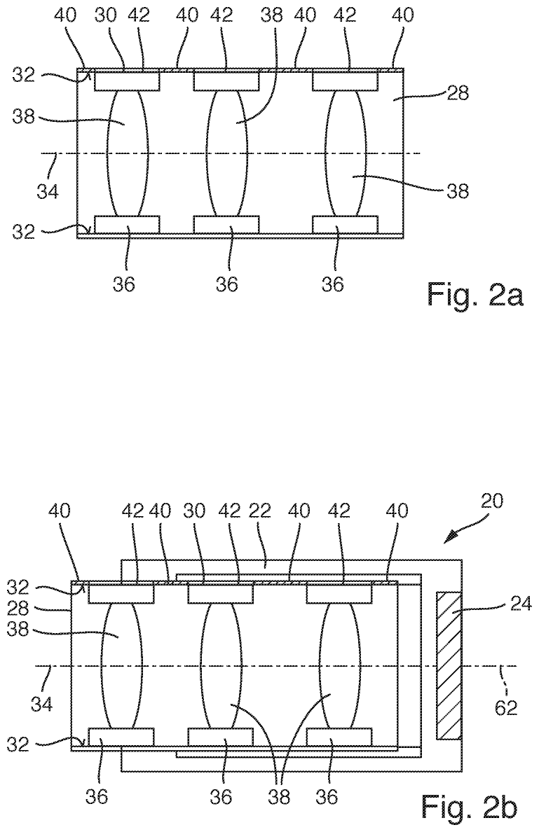

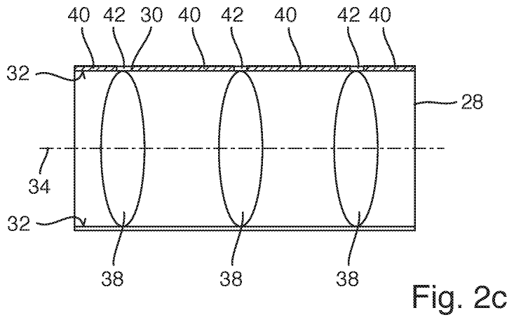

FIG. 2b shows an imaging unit 20 in a schematically simplified longitudinal sectional view with an objective tube 28 that is accommodated in a guide tube 22. In FIG. 2a, the objective tube 28 is shown in a schematic longitudinal section. The endoscope 2 shown in FIG. 1 has the imaging unit 20. The imaging unit 20 comprises an optional image sensor 24 and at least one or more optical elements, such as lenses.

A plurality of lenses 38 are arranged sequentially in the objective tube 28 in the longitudinal direction (see FIG. 2a). Each of the lenses 38 is accommodated in a centered mount 36 so that the lenses 38 are surrounded by the respective mount 36 in the peripheral direction. When the lenses 38 are arranged in the objective tube 28, the centered mount 36 causes the optical axis of the respective lens 38 to be aligned collinearly with the longitudinal axis 34, or respectively the central longitudinal axis of the objective tube 28. The centered mounts 36 are for example produced by an ultra-precise center turning method and have a precise outer diameter. The objective tube 28 has an inner lateral surface 32 so that, when introducing the mounts 36 with the centered lenses 38 into the objective tube 28, the mounts 36 are brought into contact with the inner lateral surface 32.

The objective tube 28 furthermore has a longitudinal slot 30 which extends in the longitudinal direction of the objective tube 28. After arranging the mounts 36 with their lenses 38 as optical elements, the longitudinal slot 30 is welded in the objective tube 28 at various points, for example using an energy-rich laser such as an Nd-YAG laser. In particular, the longitudinal slot 30 in the regions between the contact surfaces of the mounts 38 are welded to the inner lateral surface 32 so that the longitudinal slot 30 has a plurality of spaced weld seams 40 in the longitudinal direction, wherein the weld seams are interrupted in the region of the mounts 36.

Due to the partial welding of the longitudinal slot 30, the inner diameter of the objective tube 28 is reduced so that the lenses 38 and their respective mount 36 are clamped gap-free in the objective tube 28. The weld seams 40 are interrupted in the region of the mounts 36 as well as the lenses 38 so that the longitudinal slot 30 has slot-like through-holes 42. An adhesive such as a thread-locking fluid that can pass through gaps is introduced into the through-holes 42 in order to connect the mount 36 to the objective tube 28.

As can be seen in FIG. 2b, the imaging unit 20 has a sleeve-like guide tube 22 which is in a fixed spatial relationship to the image sensor 24, in particular a flat image sensor such as a CCD or CMOS sensor. The image sensor 24 is arranged in the guide tube 22 only as an example in the depicted exemplary embodiment. Moreover, the objective tube 28 is, or respectively can be accommodated at least sectionally in an inner chamber enclosed by the guide tube 22. The guide tube 22 and the objective tube 28 can be produced from metal, such as stainless steel.

On the end face facing away from the image sensor 24, an adhesive gap is formed between the objective tube 28 and guide tube 22 so that the guide tube 22 and the objective tube 28 are connected to each other by introducing adhesive into the adhesive gap after adjusting the objective tube 28. After adjusting the objective tube 22, the optical axes of the lenses 38 are aligned and adjusted collinearly to the longitudinal axis 34 and can also be adjusted to the optical axis of the image sensor.

In FIG. 2c, the objective tube 28 is shown in a schematic longitudinal section according to another exemplary embodiment. In this case, a plurality of optical lenses 38 are arranged sequentially in the objective tube 28 in the longitudinal direction, wherein each lens 38 is brought directly into contact with the inner lateral surface 32 of the objective tube 28. In comparison to the exemplary embodiment shown in FIG. 2a, the lenses 38 are inserted without a mount 36 (see FIG. 2a) in the exemplary embodiment in the objective tube 28 portrayed in FIG. 2c.

The objective tube 28 furthermore has a longitudinal slot 30 which extends in the longitudinal direction of the objective tube 28. After arranging the lenses 38 as optical elements, the longitudinal spot 30 is welded in the objective tube 28 at various points, for example using a laser such as an Nd-YAG laser. In particular, the longitudinal slot 30 in the regions between the lenses 38 are welded so that the longitudinal slot 30 has a plurality of spaced weld seams 40 in the longitudinal direction, wherein the weld seams 40 are interrupted in the region of the lenses 38.

By at least partially welding the longitudinal slot 30, the inner diameter of the objective tube 28 is reduced so that the lenses 38 are clamped gap-free and without an intermediate mount or the like in the objective tube 28, whereby the lenses 38 are in direct contact in a peripheral direction with the inner lateral surface 32.

The weld seams 40 are interrupted in the region of the lenses 38 so that the longitudinal slot 30 has slot-like interruptions 42, and the lenses 38 in this region of the through-holes 42 are not in direct contact with the inner lateral surface 32.

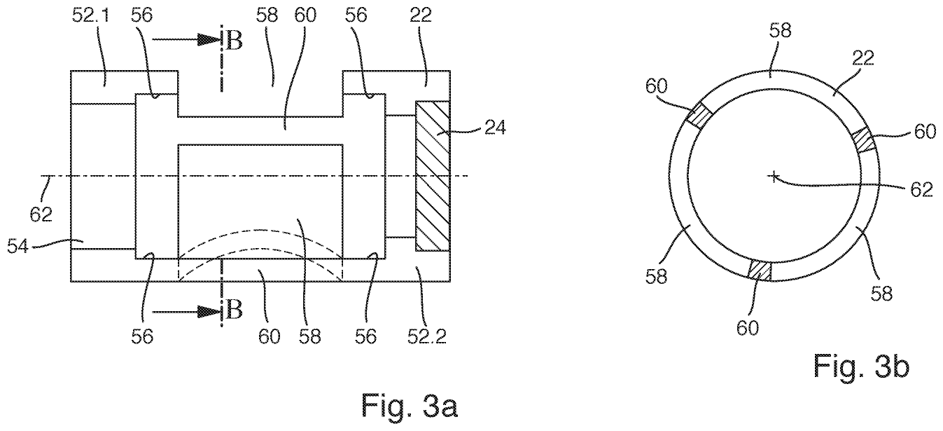

FIG. 3a schematically illustrates a longitudinal section of a guide tube 22 for accommodating an objective tube. The guide tube 22 has two tubular head ends 52.1, 52.2. The image sensor 24 is accommodated on the head end 52.2. The head end 52.1 is formed on the end face with an opening 54 so that an objective tube can be introduced or shoved through the opening 54 into the guide tube 22. The guide tube 22 has an undercut 56 between the head ends 52.1, 52.2 so that the inner diameter of the opening 54 of the head end 52.1 is smaller than the inner diameter of the undercut 56.

Furthermore, recesses 58 are provided in the region of the undercut 56 between the two head ends 52.1, 52.2, wherein a connecting bar 60 is formed in each case in the peripheral direction between two recesses 58.

FIG. 3b schematically shows a cross-section of the guide tube 22 according to the intersection line B-B drawn in FIG. 3a. The flexible connecting bars 60 by means of which the tubular head ends 52.1, 52.2 of the guide tube 22 are connected to each other are arranged evenly in the peripheral direction of the guide tube 22. The connecting bars 60 can be formed as flexible bars.

In order to bend the connecting bars 60 radially inward, or to bend them as indicated in FIG. 3a with respect to the bottom connecting bar 60, the connecting bars 60, while adjusting an objective tube (not shown in this case) that is accommodated in the interior of the guide tube 2 are for example hit with an energy-rich beam of a laser such as an Nd-YAG laser so that the connecting bars 60 are bent by being heated from the radiation energy of the laser. This brings the connecting bars into contact with the outer peripheral surface of the objective tube arranged in the interior of the guide tube 22. In this case, a tilt of the optical axis of the lens system is eliminated when using a corresponding measuring device while adjusting the objective tube, and the optical axis of the lenses is preferably adjusted collinearly to the optical axis 62 of the image sensor 24.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMBERS

2 Endoscope

4 Shaft

6 Housing

8 Eyepiece

10 Light source

12 Connecting cable

14 Camera head

16 Connection

20 Imaging unit

22 Guide tube

28 Objective tube

30 Longitudinal slot

32 Inner lateral surface

34 Longitudinal axis

36 Mount

38 Lens

40 Weld seam

42 Through-hole

52.1, 52.2 Head end

54 Opening

56 Undercut

58 Recess

60 Connecting bar

1. An imaging unit for a surgical instrument, the imaging unit comprising:
an objective tube; and
an optical element accommodated in the objective tube,
wherein one or more of:
the optical element is clamped gap-free in the objective tube;
the optical element is accommodated in a mount in the optical tube and the mount is clamped gap-free in the objective tube; and
the optical element is accommodated in a centered mount.

2. The imaging unit according to claim 1, wherein the objective tube has a through-hole in the region of the optical element, when the optical element is arranged in the objective tube in the region of the through-hole, the optical element not being brought into contact with an inner lateral surface of the objective tube.

3. The imaging unit according to claim 1, wherein the objective tube has a through-hole in one or more of the region of the optical element and in the region of the mount, wherein when the mount with the optical element is arranged in the objective tube in the region of the through-hole, the mount of the optical element is not brought into contact with an inner lateral surface of the objective tube.

4. The imaging unit according to claim 2, wherein the through-hole is formed in the objective tube as one of a gap and a slot.

5. The imaging unit according to claim 3, wherein the through-hole is formed in the objective tube as one of a gap and a slot.

6. The imaging unit according to claim 2, wherein the through-hole in the objective tube for the optical element is formed between two slot connecting regions of the objective tube relative to the longitudinal extension of the objective tube.

7. The imaging unit according to claim 3, wherein the through-hole in the objective tube for the optical element is formed between two slot connecting regions of the objective tube relative to the longitudinal extension of the objective tube.

8. The imaging unit according to claim 6, wherein at least one of the two slot connecting regions is formed as a weld seam.

9. The imaging unit according to claim 7, wherein at least one of the two slot connecting regions is formed as a weld seam.

10. An imaging unit for a surgical instrument, the imaging unit comprising:
an objective tube accommodating at least one optical element; and
a sleeve tube with two head ends for accommodating the objective tube;
wherein the two head ends of the sleeve tube are formed tubular, and the two head ends of the sleeve tube are connected to each other by at least one connecting bar.

11. The imaging unit according to claim 10, wherein the head ends of the sleeve tube are connected to each other by a plurality of connecting bars.

12. The imaging unit according to claim 11, wherein one of a gap and a recess is formed in a peripheral direction of the sleeve tube between two adjacent connecting bars of the plurality of connecting bars.

13. The imaging unit according to claim 12, wherein a width of one or more of the plurality of connecting bars is greater in the peripheral direction of the sleeve tube than a width of a space or a width of a recess between the two adjacent connecting bars of the plurality of connecting bars.

14. The imaging unit according to claim 10, wherein the at least one connecting bar is flexible or curved in a radial direction relative to a longitudinal axis of the sleeve tube.

15. A surgical instrument comprising the imaging unit according to claim 1.

16. A surgical instrument comprising the imaging unit according to claim 10.

17. A method for manufacturing an imaging unit for a surgical instrument, wherein the imaging unit comprises a sleeve tube and an objective tube arranged in the sleeve tube, wherein an optical element is accommodated in the objective tube, the method comprising:
one of inserting the optical element into the objective tube provided with a longitudinal slot, or where the optical element is accommodated in a mount in the objective tube, inserting the mount into the objective tube provided with the longitudinal slot,
subsequent to the inserting, reducing the diameter of the objective tube to clamp one of the optical element or the mount of the optical element in the objective tube, and
subsequent to the reducing, introducing the objective tube into the sleeve tube (22).

18. The method according to claim 17, wherein the reducing comprises at least partially welding the longitudinal slot in a longitudinal direction of the objective tube.

19. The method according to claim 18, wherein the welding comprises partially welding the longitudinal slot so as not to weld the longitudinal slot in a region corresponding to a longitudinal position of the optical element or so as not to weld the longitudinal slot in a region corresponding to a longitudinal position of the mount of the optical element.
